(19) United States
(12) Patent Application Publication
(10) Pub. No.: US 2012/0123279 A1
(43) Pub. Date: May 17, 2012

(54) METHOD AND APPARATUS FOR THE ANALYSIS OF A BALLISTOCARDIOGRAM SIGNAL

(75) Inventors: Christoph Brueser, Troisdorf (DE); Kurt Stadlthanner, Fuerth (DE); David Friedrich, Aachen (DE); Andreas Brauers, Iserlohn (DE)

(73) Assignee: KONINKLIJKE PHILIPS ELECTRONICS N.V., EINDHOVEN (NL)

(21) Appl. No.: 13/386,746

(22) PCT Filed: Jul. 23, 2010
(86) PCT No.: PCT/IB2010/053364

§ 371 Date: Jan. 24, 2012

(30) Foreign Application Priority Data
Jul. 31, 2009 (EP) 09166924.2

(51) Int. Cl. A61B 5/02

(52) U.S. Cl. 600/481

(57) ABSTRACT

There is provided a method and apparatus for the analysis of a ballistocardiogram signal. The method comprises detecting heart beats in the BCG signal by locating typical features of a heart beat for a user in the BCG signal, the typical features of the heart beat having been obtained during a training step.

### TECHNICAL FIELD OF THE INVENTION

The invention relates to a method and apparatus for the analysis of a ballistocardiogram signal, and in particular to a method and apparatus that provides for the detection of single heart beat events in a ballistocardiogram signal.

### BACKGROUND TO THE INVENTION

A ballistocardiograph (BCG) measures the movement of the human body due to the momentum of the blood as it is pumped by the heart.

The BCG has advantages over the electrocardiograph (ECG) in that the measurement of body vital signs is possible without electrodes having to be glued to the body or for special sensors like belts, textiles or the like to be worn. Due to this unobtrusive nature the BCG is best suited for monitoring the heart activity of people at night over a long period of time. Likewise, BCG systems can be used as an additional safety measure to monitor patients in the general ward of hospitals without reducing their sleep quality. Since modern BCG systems can be fully integrated into a bed and can be activated by a single switch, the additional safety provided by such a system requires minimal extra effort from healthcare professionals.

Currently, algorithms for analysing ballistocardiogram signals to determine the heart rate use spectral methods or methods in the time domain that detect the reoccurrence of certain patterns by, for example, evaluating the autocorrelation function of the signal. In all of these approaches, segments of the signal have to be considered which last for several seconds such that they cover multiple heart beats. As a result, average heart beats over a period of time are obtained, but no beat-to-beat information is available.

Some algorithms for beat-to-beat estimation from ballistocardiogram signals have been presented, but these either require a large and expensive sensor array in order to work properly (“FFT averaging of multichannel BCG signals from bed mattress sensor to improve estimation of heart beat interval” by Kortelainen, J. M. and Virkkala, J., Engineering in Medicine and Biology Society, 2007, EMBS 2007, 29th Annual International Conference of the IEEE, 22-26 Aug. 2007, pages 6685-6688), human interaction (“Automatic Ballistocardiogram (BCG) Beat Detection Using a Template Matching Approach” by J. H. Shin, B. H. Choi, Y. G. Lim, D. U. Joeng and K. S. Park, Engineering in Medicine and Biology Society, 2008, EMBS 2008, 30th Annual International Conference of the IEEE, 21-24 Aug. 2008) or use different sensor modalities and lack accuracy (“Estimation of Respiratory Waveform and Heart Rate Using an Accelerometer” by D. H. Phan, S. Bonnet, R. Guillemaud, E. Castelli, N. Y. Pham Thi, Engineering in Medicine and Biology Society, 2008, EMBS 2008, 30th Annual International Conference of the IEEE, 21-24 Aug. 2008).

It is questionable whether these algorithms can be brought to market, or whether they are able to deal with the high intra- and inter-patient variability of ballistocardiogram signals, particularly those patients with arrhythmias.

Arrhythmias are a widespread problem and can be a predictor of critical health conditions. According to the American Heart Association an estimated 2.2 million Americans are living with atrial fibrillations. This means that the assumption made by some algorithms of a regular beating heart does not hold for a significant fraction of the people to be monitored in the general wards of hospitals, for instance.

Depending on the severity of arrhythmias, existing algorithms that try to detect heart beats in BCG signals fail sooner or later since they usually assume that the heart is beating rather regularly. Thus, there is a need for an algorithm for processing ballistocardiogram signals that is not based on this regularity assumption and which is thus able to work also in the case of severe arrhythmia. It is also desirable for an algorithm that can provide a reliable beat to beat analysis of the BCG signal.

Furthermore, many of the existing BCG analysis algorithms only work offline. Thus, it is desirable for the algorithm to be suitable for use online (i.e. continuously) and therefore suitable for monitoring and alerting tasks in hospitals, nursing homes or at home.

### SUMMARY OF THE INVENTION

According to a first aspect of the invention there is provided a method of detecting heart beats of a user in a ballistocardiogram, BCG, signal, the method comprising detecting heart beats in the BCG signal by locating typical features of a heart beat for the user in the BCG signal, the typical features of the heart beat having been obtained during a training step.

According to a second aspect of the invention, there is provided an apparatus for use with a device for measuring a ballistocardiogram signal of a user, the apparatus comprising means for receiving a ballistocardiogram signal from the device; and processing means for performing the method described above on the received ballistocardiogram signal.

According to a third aspect of the invention, there is provided a computer program product comprising computer program code that, when executed on a computer or processor, is configured to cause the computer or processor to perform the method described above.

### BRIEF DESCRIPTION OF THE DRAWINGS

The invention will now be described in the following detailed description, by way of example only, with reference to the following drawings, in which:

FIG. 1 is a flow chart illustrating the principle of detecting heart beats according to the invention;

FIG. 2 is a graph illustrating an unfiltered ballistocardiogram signal;

FIG. 3 is a flow chart illustrating a method of training the algorithm according to the invention;

FIG. 4 is a graph illustrating the result of filtering the ballistocardiogram signal to remove the breathing component;

FIG. 5 is a graph illustrating the result of smoothing the filtered ballistocardiogram signal;

FIG. 6 is an illustration of the parameters derived from the ballistocardiogram signal in accordance with an embodiment of the invention;

FIG. 7 is a flow chart illustrating the implementation of step 107 in FIG. 3 in more detail;

FIG. 8 is a graph illustrating the high frequency filter response envelope of the ballistocardiogram signal;

FIG. 9 is a flow chart illustrating the method of using the algorithm to detect heart beats in a ballistocardiogram signal;

FIG. 10 is a graph illustrating reliability score triplets;

FIG. 11 is a graph illustrating the effectiveness of the algorithm in relation to heart beats detected using an ECG;

FIG. 12 is a graph comparing the beat to beat intervals determined by the algorithm and by using an ECG;

FIG. 13 is a graph illustrating the effectiveness of the algorithm in detecting a heart beat when arrhythmia is present in relation to an ECG; and

FIG. 14 is a block diagram of an apparatus in accordance with an embodiment of the invention.

### DETAILED DESCRIPTION OF THE PREFERRED EMBODIMENTS

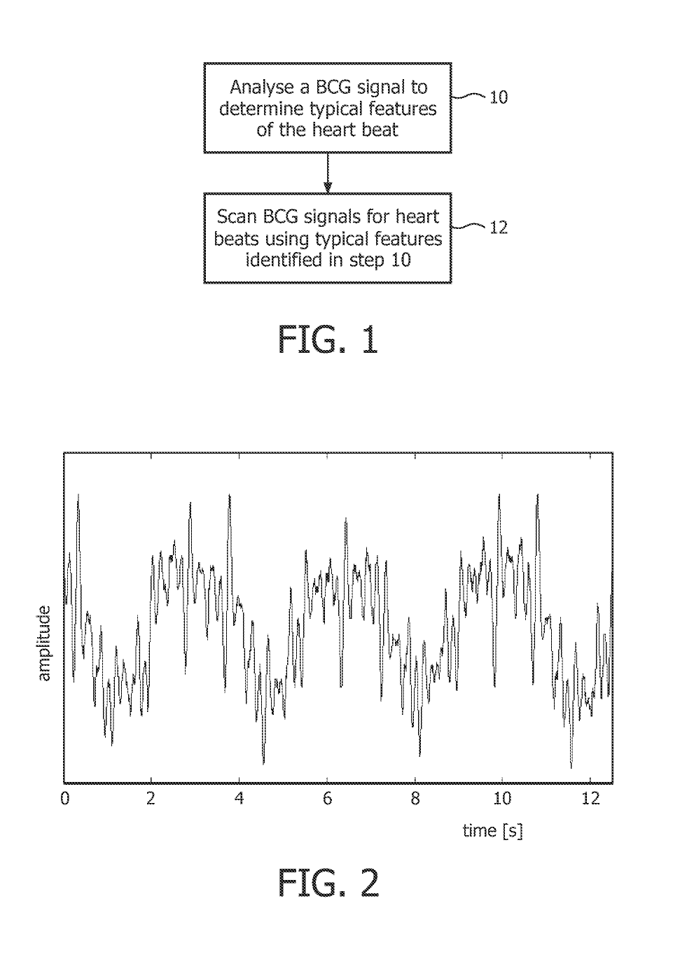

The flow chart in FIG. 1 illustrates the general principles used by the algorithm according to the invention. In the first step (step 10), the training step, a short segment of a ballistocardiogram (BCG) signal is analyzed and the typical features of the heart beat are determined. In the second step (step 12), the assignment step, newly recorded samples of a live BCG signal are scanned for heart beats, or, in other words, the algorithm tries to rediscover the features learned in the training step in the newly recorded signals. The operation of the training and assignment steps are briefly outlined below.

In order to learn the typical features of the heart beat during the training period (step 10), the segment of the ballistocardiogram used for the training is first filtered in order to remove any breathing components and to smooth the signal. Specific points are identified in the resulting signal which can be used to parameterize the signal. At each specific point the next n parameters are used to form a feature vector. The resulting feature vectors are clustered by similarity. Here, the principle is that the feature vectors covering individual heart beats will be similar enough that they will be clustered together, while other feature vectors will be found in different clusters.

Alongside this processing, the original (unfiltered) training segment is filtered in order to remove the breathing signal, but this time, no smoothing occurs. In this signal, high frequency components are determined which usually coincide with each heart beat. In this process, the passband frequencies of the filter used to detect the high frequency components are adjusted automatically.

The cluster that contains the feature vectors relating to the heart beats is identified using the characteristics of the individual clusters together with the determined high frequency components.

During the assignment step (step 12) the newly measured or received signal is again filtered, smoothed and parameterized as described above. The parameters of the signal are compared with the cluster that was identified as containing the features of the heart beats in the training step (step 10). Furthermore, high frequency components are detected in the newly arriving signal. Based on the similarity to the cluster containing the heart beats and the location of the high frequency components, a first estimate of the locations of heart beats is made. The positions of heart beats given by the first estimate are calibrated in a refinement step which is based on an autocorrelation between two consecutive heart beat signals.

The operation of the algorithm according to the invention will now be described in more detail with reference to FIGS. 2 to 10 of the accompanying drawings.

FIG. 2 shows a typical segment of a BCG signal consisting of a breathing component which expresses itself as a low frequency quasi-sinusoidal signal plus a heart beat component which leads to sharp bursts. The goal of the presented algorithm is to identify the bursts related to the heart beat and to determine the pulse-to-pulse (otherwise known as the beat-to-beat) time distance between them. For this purpose, as summarised above, the algorithm first detects and learns the characteristic features of the heart beat.

It will be appreciated that these features change from individual to individual and also depend strongly on the position of the monitored person relative to the BCG sensor.

### Learning Step

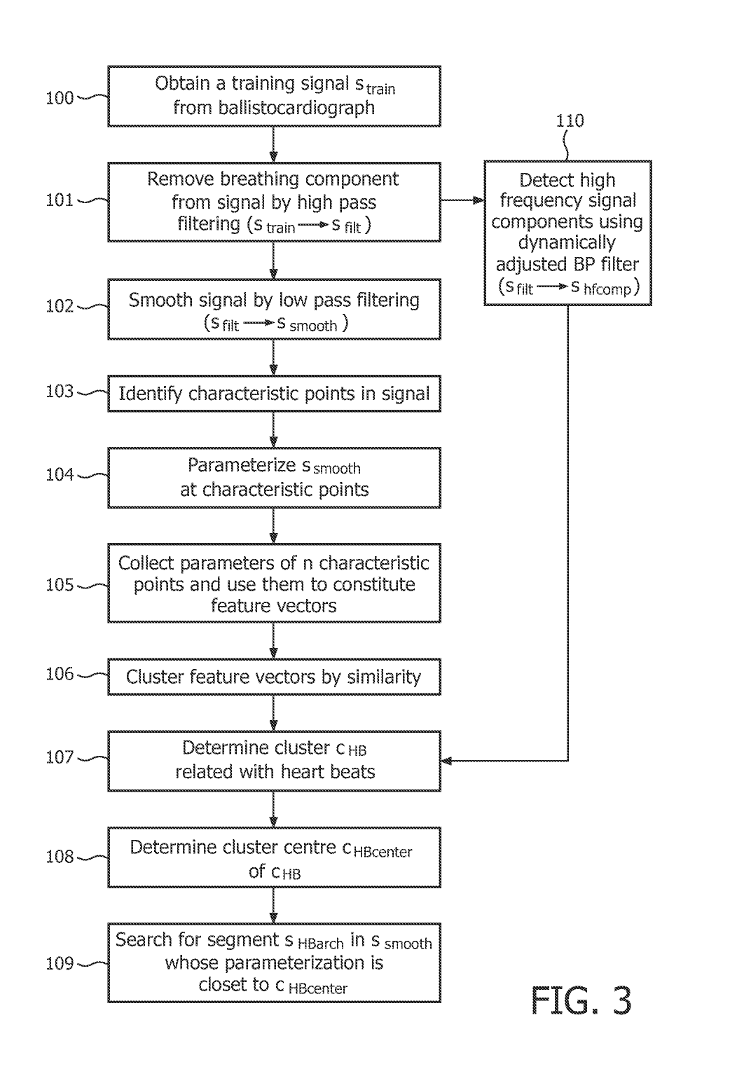

The learning step (step 10) of the algorithm is depicted in FIG. 3. In step 100, a training signal, denoted sₜᵣₐᵢₙ comprising a segment of a ballistocardiogram signal is obtained. The training segment must be long enough such that it contains a number of heart beats, and it must be clear of artefacts. This segment is preferably between 10 and 60 seconds long.

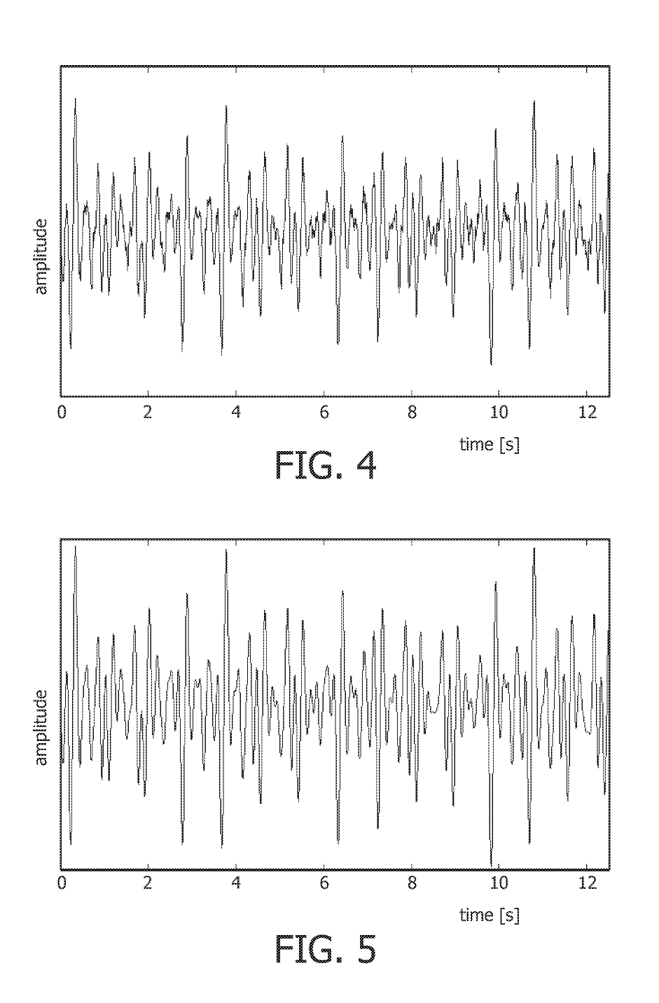

In step 101, the breathing component is removed from the ballistocardiogram signal by means of a high pass filter with a cut-off frequency of 1 Hz. The resulting filtered signal s_{filt} (depicted in FIG. 4) is used in two different processing paths, as described further below.

In the later steps of the algorithm every local extremum (i.e. maximum or minimum) of the pre-processed signal is analyzed. In these steps only meaningful, well-expressed extrema should be taken into account, while considering negligibly small extrema hampers the success of the algorithm. For this reason, the segment is first smoothed by means of a low pass filter with a cut-off frequency of 10 Hz (step 102 in the flowchart of FIG. 3) in the first path of the algorithm (steps 102 to 106). FIG. 5 shows the smoothed signal, Sₛₘₒₒₜₕ. As can be seen, high frequency noise and hence also small local extrema still visible in FIG. 4 are removed such that only the basic shape of the signal remains.

In the BCG signal sₛₘₒₒₜₕ the heart beats are discernible as groups of peaks with similar amplitudes and inter-peak-distances after the breathing component has been removed.

In step 103, characteristic points the filtered and smoothed BCG signal are detected. In a preferred embodiment, these characteristic points are the maximas and the minimas of the signal.

In step 104 each detected local maxima in the signal sₛₘₒₒₜₕ is parameterized. In a preferred embodiment, each local maxima k is parameterised using the following features:
i) the amplitude (aₘₐₓ) of the maxima,
ii) the distance (dₘₐₓ) between the local maxima and the next local minima to the right,
iii) the amplitude (aₘᵢₙ) of the next local minima to the right of the local maxima, and
iv) the distance (dₘᵢₙ) between the next local minima to the right of the local maxima and the next local maxima to the right.

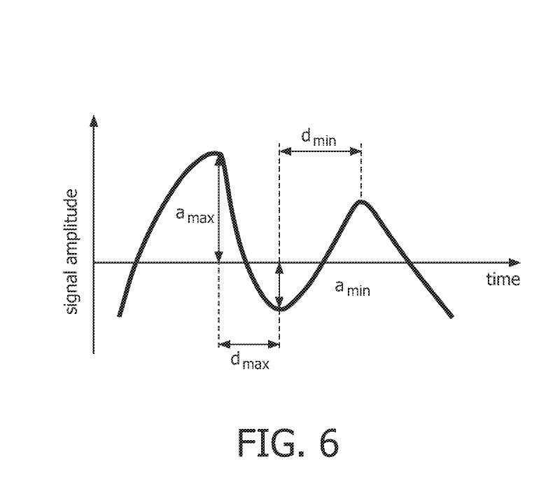

These parameters are illustrated in FIG. 6 and they give a rough description of the shape of the signal.

In step 105 the parameters of n (with n usually in the range n=3 to 10) consecutive peaks are collated and used to form feature vectors fₖ. A feature vector fₖ is given by

fₖ=[a_{max,k}, d_{max,k}, a_{min,k}, d_{min,k}, a_{max,k+1}, d_{max,k+1}, a_{min,k+1}, d_{min,k+1}, . . . , a_{max,k+n-1}, d_{max,k+n-1}, a_{min,k+n-1}, d_{min,k+n-1}] (1)

where k=1, 2, . . . , K−n+1, and K denotes the number of peaks in the training segment sₜᵣₐᵢₙ. The number of peaks n to be collated into each feature vector fₖ roughly corresponds to the number of peaks which appear during each heart beat in the BCG signal. Thus, step 105 results in K−n+1 distinct feature vectors fₖ.

It will be appreciated by those skilled in the art that other parameterizations of the BCG signal sₛₘₒₒₜₕ are possible. For instance, the signal can be approximated by splines where the knots of the spline approximation are the local maxima and minima and possibly a low number of other points (usually only one) in-between them. Alternatively, parameterization of the signal in terms of its most significant Fourier coefficients is possible.

In step 106, the feature vectors fₖ are clustered by similarity. Prior to this, in some embodiments of the invention, as clustering methods usually work best in low rather than high dimensional vector spaces, the dimension of the feature vectors can be reduced by means of principal component analysis.

In the BCG signal sₛₘₒₒₜₕ, the heart beats are visible as repetitive patterns of peaks. Therefore, the particular feature vectors that describe the heart beats will be similar and will be distinguished considerably from those feature vectors that describe random fluctuations of the signal in-between the beats.

Therefore, in step 106, grouping the feature vectors by similarity will lead to a cluster or clusters that contain the feature vectors related to the heart beats only.

Those skilled in the art will appreciate that various methods, like, for instance, k-means clustering, hierarchical clustering, self organizing maps and the like are suitable for grouping the feature vectors fₖ into clusters. It will also be appreciated that various distance measures d, like, for instance, the Euclidian distance, the angle between the individual feature vectors etc. can be used in the clustering step.

In a preferred embodiment of the invention, apart from the standard clustering methods, it is of advantage to form a cluster for each feature vector that contains, each time, the P (where P is in the region of 10) next neighbours with respect to a certain distance measure (preferably the Euclidian distance).

Any of the clustering techniques leads to a number of clusters M, and it is necessary to identify the cluster that contains the feature vectors describing the heart beat patterns (step 107).

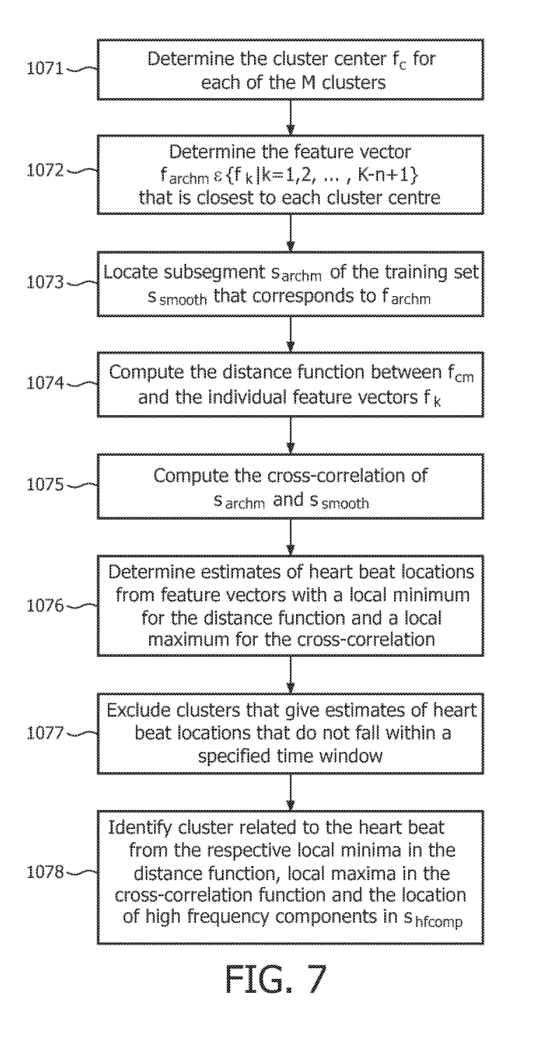

The flow chart in FIG. 7 illustrates the steps carried out in step 107.

In step 1071, for each of the M clusters, the cluster centre f_{cm} is determined (where m indicates the m-th cluster and m=1, . . . , M). Techniques for determining cluster centres are well known to those skilled in the art, and will not be described further herein.

In step 1072, for each of the M cluster centres identified in step 1071, the feature vector fₖ in the set {fₖ|k=1,2, . . . ,K−n+1} that is closest to the cluster centre f_{cm} is identified. This feature vector is denoted as f_{archm}, as it is the feature vector that is the archetype or model of the part of the signal represented by the m^{th} cluster. Thus, for the cluster that represents the heart beat signal, the archetype feature vector f_{arch} or the cluster centre f_{c} for that cluster can be seen as a model for a parameterised heart beat.

Next, in step 1073, the training signal sₛₘₒₒₜₕ is searched for the subsegment s_{archm} that best corresponds to f_{archm}. Effectively, steps 103 and 104 of the learning procedure are reversed for the feature vector f_{archm}). Thus, the located subsegment s_{archm} contains those n peaks of sₛₘₒₒₜₕ whose parameterization is closest to f_{archm}.

Steps 1074-1078 described below indicate how the cluster and cluster centre representing the heart beat signal are identified from the M subsegments s_{arch} and M cluster centres identified in step 1072. Although f_{c} is used in the following steps, it will be appreciated that f_{arch} could be used instead.

Firstly, in step 1074, the distance function between each cluster centre f_{cm} and the individual feature vectors fₖ is computed. Preferably, the same distance measure as used in the clustering step (step 106) is used.

This distance function d will have a local minima with respect to f_{cm} at position p, where p ε [1,2, . . . ,K−n+1] when the following condition is fulfilled: d(f_{c}, fₚ₋₁)>d(f_{c}, fₚ)<d(f_{c},fₚ₊₁)

It will be appreciated that the distance function d between f_{cm} and any feature vector related to or that is part of that cluster will have a local minimum.

Then, in step 1075, the cross correlation between each subsegment s_{archm} and the training signal sₛₘₒₒₜₕ is determined. It will be appreciated that the results of cross correlation will have maxima where the subsegment s_{archm} is most similar to the training signal sₛₘₒₒₜₕ (i.e. the parts of the signal that the feature vectors in the mₜₕ cluster represent).

Considering the cluster and cluster centre that represents the heart beat (which have still not been identified from the M clusters at this stage), there will be a local minimum in the distance function and a local maximum in the cross correlation at each heart beat in the BCG signal sₛₘₒₒₜₕ.

Thus, in step 1076, first estimates of heart beat locations are determined separately for each of the M clusters and cluster centres using the minima of the distance function and the maxima of the cross correlation function. Further details about how these two functions are used to identify locations is given in the assignment section below.

In addition to the cluster and cluster centre relating to the heart beat signal providing locations for the heart beats, the other clusters will also identify locations in sₛₘₒₒₜₕ having local minima in the distance function and local maxima in the cross correlation function. However, these local extrema generally only appear sporadically in the BCG signal.

Hence clusters that are not related to the heart beat can be identified by checking the time between two individual local extrema in the BCG signal (step 1077). If the intervals between consecutive “heart beats” are larger than say, 3 seconds and/or smaller than 0.25 seconds then it can be assumed that the corresponding cluster is not related with the heart beat and can be ignored in the further analysis.

Although the check in step 1077 above is useful in excluding many of the clusters from the analysis, it is often the case that more than one cluster does pass the check. Hence, a further knock-out criterion is needed.

It is a general observation that heart beats lead to segments with comparatively high frequencies in the BCG. In the literature (“Higher Frequency Phenomena in the Normal Ballistocardiogram” by J. N. Edson, R. Dickes, G. H. Flamm and M. Tobin, Circulation Research, 1958, Vol. 1, pages 405-409) these high frequency components are said to be related with the mechanical forces caused by the opening and closing of the cardiac valves. Since the frequencies of these components can vary individually but also depending on the location of the BCG sensor, a dynamically adjusted bandpass filter is used to detect them in this method (see step 110 in FIG. 3).

The optimal parameters are determined by filtering the signal segment s_{filt} (from step 101 in FIG. 3) with various bandpass filters. The set of bandpass filters is constructed so that each individual filter has a bandwidth of 2 Hz and the whole set covers the frequency range from 4 Hz up to 22 Hz in 0.1-0.5 Hz increments. Thus, the set of bandpass filters can comprise filters having cut-off frequencies ={[4 Hz, 6 Hz], [4.1 Hz, 6.1 Hz], [4.2 Hz, 6.2 Hz], . . . , [20 Hz, 22 Hz]}).

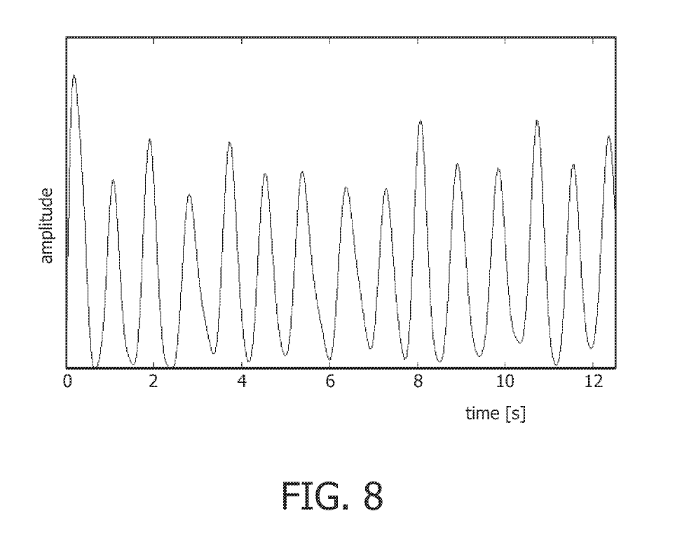

For each filter in the set, the filter response is squared and a lowpass filter with a cut-off frequency of 3.5 Hz is applied to obtain a filter response envelope (as shown in FIG. 8).

For each of the filter response envelopes, a relevance score is assigned to each peak in the envelope by means of
a) the ratio between the amplitude of the peak and the average peak amplitude plus peak amplitude standard deviation, and
b) the symmetry of the peaks.

The filter which leads to the highest average relevance score is then selected and used in step 110 to detect the high frequency components of the filtered BCG signal s_{filt}. A more detailed description of the relevance score computation is provided in the assignment section below.

The high frequency components detected in step 110 using the optimal filter can now help to identify the cluster related to the heart beat in step 107. Returning now to FIG. 7, and specifically step 1078, the cluster whose local minima in the distance function and the local maxima in the correlation function coincide best with the location of the high frequency components determined in step 110 is considered as being best related to the heart beat. Additionally, the depth of the local minima of the distance function, the amplitude of the local maxima of the cross-correlation function as well as the amplitude of the maxima of the high frequency components can be taken into account in order to determine the cluster best describing the heart beat pattern. This cluster is denoted c_{HB} herein. All other clusters remaining in the analysis at this stage are now disregarded.

The cluster centre f_{c} of this cluster is now denoted c_{HBcentre} (step 108 in FIG. 3) and the segment in sₛₘₒₒₜₕ that most closely matches c_{HBcentre} is identified. This segment is denoted s_{HBarch} (step 109). This terminates the learning part of the algorithm.

### Assignment Step

Once the centre c_{HBcentre} and the corresponding archetype of a heart beat signal s_{HBarch} have been determined, newly incoming BCG signals (in the online case) or the rest of the BCG signal (in the offline case) can be scanned for heart beats (step 12 in FIG. 1). In the following, the online case will be described. Those skilled in the art will appreciate that analogous considerations also hold for the offline case.

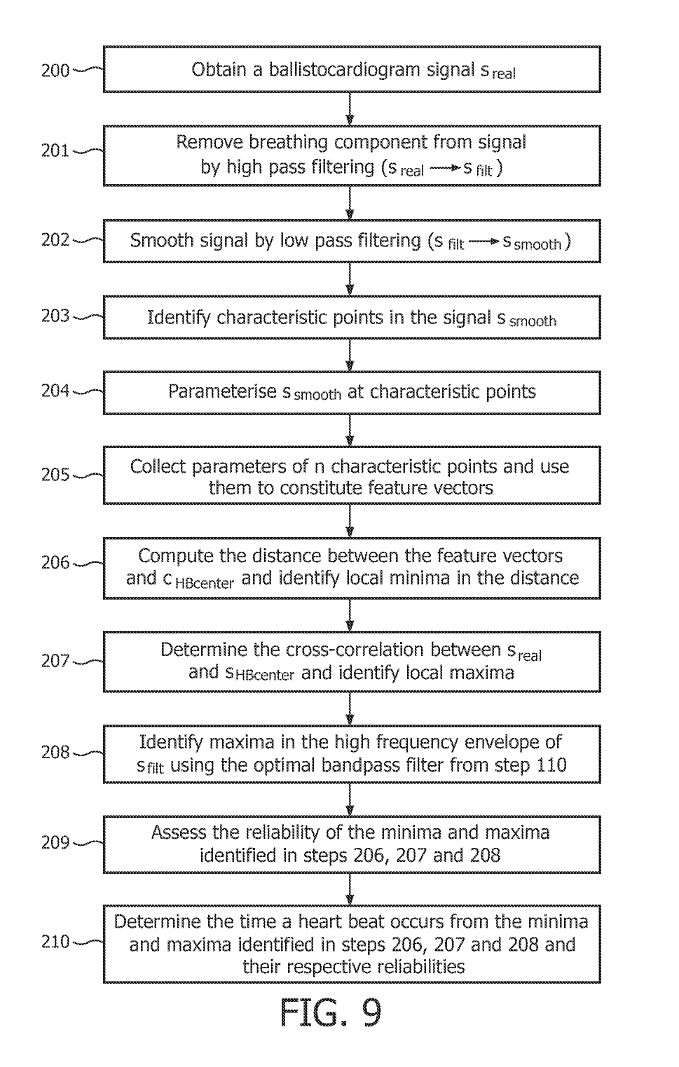

FIG. 9 illustrates the method used during the assignment step.

Whenever new samples (Sᵣₑₐₗ) come in from the BCG sensor they are filtered (step 201) and smoothed (step 202) as in steps 101 and 102 of the training algorithm shown in FIG. 3. Likewise , characteristic points in the filtered and smoothed signal (i.e. local maxima and minima) are determined (step 203, which is the analogue of step 103). The signal is then parameterized at the characteristic points and feature vectors are constituted (steps 204 and 205).

The distances between the feature vectors and the heart beat cluster centre c_{HBcentre} are computed and scanned for local minima in step 206 (which is analogous to step 1074 in FIG. 7). Likewise, the cross correlation and its maxima between s_{HBarch} and the newly incoming signals (after filtering and smoothing) are determined (step 207).

Further the optimal bandpass filter determined in step 110 of the learning algorithm is applied to scan the new BCG signal (s_{filt}) for maxima in the high frequency envelope (step 208).

The results of the analysis in steps 206, 207 and 208 can be used to identify where heart beats occur in the BCG signal sᵣₑₐₗ. However, it will be noted that the location of the minimum of the distance function, the maximum of the cross correlation and the maximum of the high frequency components might appear at slightly different locations (times). Additionally spurious high frequency components, but also wrong local extrema of the distance and cross correlation functions can appear. In such cases it is important to assess the reliability of the individual criteria in order to find a reasonable compromise between them. Thus, in step 209, the reliability of the minima and maxima found in steps 206, 207 and 208 is assessed.

The reliability of a local minimum of the distance function is evaluated by means of its depth relative to the height of the two neighbouring maxima. For this purpose the differences in amplitude between the left maxima and the minima (hₗ) and between the right maxima and the minima (hᵣ) are computed. The reliability r_{d} of a minimum with amplitude aₘᵢₙ is then evaluated as

r_{d}=(hₗ+hᵣ)/(2(aₘᵢₙ+max(hᵣ,hₗ)))

It can be shown that r_{d} is normalized (i.e. 0≦r_{d}≦1) by construction. The maximal score of 1 is assigned to minima which are symmetric with respect to the height of their neighbouring peaks and have an amplitude of 0.

Likewise a cross correlation peak with amplitude aₘₐₓ can be assessed using rₓ=(hₗ+hᵣ)/(aₘₐₓ)

where h₁ and hᵣ are the amplitude differences between the peak and the left and right minima respectively. As with r_{d}, rₓ is normalized and the maximal score is assigned to peaks that are symmetric with respect to their height above the left and right minima.

Finally, the reliability of a peak in the high frequency components of the signal is evaluated using

r_{hf}=(hₗ+hᵣ)/(2aₘₐₓ)(aₘₐₓ/a_{ref})

In this case, the first factor of the formula is identical to the calculation of rₓ and thus quantifies the peak's relative symmetry, whereas the second factor adjusts the score by the ratio of the peak amplitude aₘₐₓ to a reference amplitude a_{ref}. The reference amplitude a_{ref} is calculated during the training sequence as the average amplitude of the high frequency peaks plus the amplitudes' standard deviation.

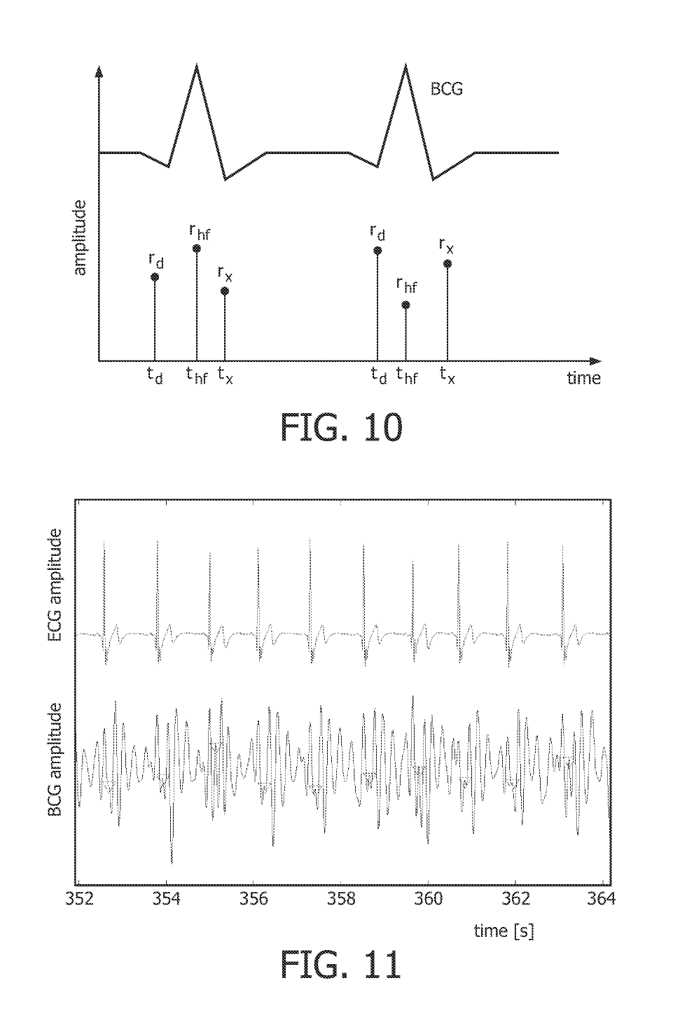

Eventually it has to be determined how the information about the reliabilities rₓ, r_{d}, and r_{hf} computed above can be combined advantageously in order to determine at what point in time a heart beat has actually occurred (step 210). The time points at which the cross correlation criterion, the distance criterion and the high frequency component criterion would detect, each by itself, a heart beat are denoted by tₓ, t_{d}, and t_{hf} respectively. In the ideal case, these three times would be identical (i.e. tₓ=t_{d}=t_{hf}). However, in real world settings they slightly differ from each other because they try to detect heart beats by focusing on different features of the BCG signal. If a heart beat is well detected by all three criteria (distance, cross-correlation and high frequency component) simultaneously, the times t_{d}, tₓ and t_{hf} will form clearly discernable triplets (see FIG. 10). The aim of the following procedure is to identify these triplets and to replace them by a representative value which can be used to identify the individual heart beats.

Consider, without loss of generality, the time t_{hf} as a starting point for the further description of the algorithm. At this time t_{hf} the high frequency criterion suggests a heart beat. In that case the algorithm detects if tₓ and t_{d} lie in the interval [t_{hf}−c, t_{hf}+c] where c is a constant usually set to about 0.3 seconds. If tₓ and t_{d} lie in this interval a representative value R_{hf}(r*_{hf},t*_{hf}) of the three times tₓ, t_{d}, and t_{hf} and of the three reliabilities rₓ, r_{d}, and r_{hf} is constructed which is defined by a point in time t*_{hf} and by a reliability r*_{hf}. The representative time t*_{hf} is computed as the sum of the times tₓ, t_{d}, and t_{hf} weighted with the corresponding reliabilities rₓ, r_{d}, and r_{hf} as follows:

t*_{hf}=(rₓtₓ+r_{d}t_{d}+r_{hf}t_{hf})/(rₓ+r_{d}+r_{hf})

The representative reliability is simply computed as the sum of the reliabilities of the cross correlation, distance and high frequency criterion, i.e.

r_{hf}=(rₓ+r_{d}+r_{hf})/N N=3

Until now it has been assumed that the distance and cross correlation criterion lead to only one time t_{d} and tₓ, respectively, in the time interval [t_{hf}−c, t_{hf}+c]. If only one or even neither of these times are found in this interval, the procedure still works as described above, however the reliabilities of the missing time points are set to zero in the above formulae. Accordingly, the value of N is set to the number of non-zero reliabilities.

Likewise, it is also possible that the distance and/or correlation criterion suggest more than one heart beat in the interval [t_{hf}−c, t_{hf}+c]. If it is assumed that the distance criterion indicates two heart beats at times t_{d1} and t_{d2} in the interval [t_{hf}−c, t_{hf}+c], then the corresponding reliabilities r_{d1} and r_{d2} are weighted by a symmetric window function centred at t_{hf} which decreases linearly or nonlinearly with the distance from t_{hf}. In a preferred embodiment, a Gaussian window function is used which decreases to (almost) zero at the ends of the interval [t_{hf}−c, t_{hf}+c]. Only the time point t_{d1} or t_{d2} which has the largest weighted reliability is considered in the following steps of the algorithm while the other one is discarded.

So far, the procedure described above was based on the time point t_{hf} as a starting point. This starting point was chosen arbitrarily, and preferably in practice the procedures outlined above are repeated with t_{d} and tₓ, respectively, as the starting points. This will lead to new representatives R_{d}(t*_{d}, r*_{d}) and Rₓ(t*ₓ, r*ₓ). In the case of clearly discernable triplets as shown in FIG. 10, the time components of the representatives t*_{d}, t*ₓ and t*_{hf} are identical.

For each time point t at which a heart beat is thought to have occurred, the algorithm computes a score. If no representative is found at time t (i.e. no representative has a time component t*_{d}, t*ₓ, or t*_{hf} that equals t) the score is zero. Otherwise, the score equals the sum of the reliability components of those representatives that have a time component equalling t.

For instance, assume that there are three representatives R_{d}(t*_{d},r*_{d}), Rₓ(t*ₓ,r*ₓ) and R_{hf}(t*_{hf},r*_{hf}) with t*_{d}=t*ₓ=t*_{hf}=t then the corresponding score S(t) at time t equals

S(t)=r*_{d}+r*ₓ+r*_{hf}

If S(t) is larger than a predefined threshold Sₜₕᵣₑₛₕ then the algorithm detects a heart beat at time t. Otherwise no heart beat is detected and the algorithm waits for the next samples (in the online case). Those skilled in the art will appreciate that the threshold Sₜₕᵣₑₛₕ can be adjusted during the training and/or assignment step depending on the reliability values of the q last detected heart beats.

Furthermore, in cases where the monitored person shows a rather regular heart beat, the recently determined beat-to-beat intervals can be used to predict when the next heart beat is most likely to occur. Around these points in time the threshold Sₜₕᵣₑₛₕ is lowered in order to reduce the number of missed beats.

The points identified above already indicate well where the individual heart beats occurred in the BCG signal. However, in order to determine the individual beat-to-beat time intervals even more precisely, a further refinement step is required. In this step the period of time over which the pattern of a peak repeats itself for the first time in the signal is fine-tuned.

In particular, let t₁, t₂, . . . , t_{N}, where N is the number of heart beats found so far, be the time points at which heart beats have been detected. Further, let s(tₚ,tₚ₊₁) denote the segment of the signal s_{filt} recorded between the time points tₚ and tₚ₊₁, with 1≦p≦N−1.

In order to fine-tune when the p-th peak repeats itself in the signal, the argument t_{shift}

t_{shift}=arg max_{dt} xcorr(s(tₚ, tₚ₊₁), s(tₚ₊₁+dt, 2tₚ₊₁−tₚ+dt)), dt ε [−0.15 s, 0.15 s]

which maximizes the cross correlation xcorr between the segment s(tₚ,tₚ₊₁) and the following segment s(tₚ₊₁+dt, 2tₚ₊₁−tₚ−1+dt) is determined. The beat-to-beat interval i_{p,p}+1 between the p-th and the p+1-th peak can eventually be computed as

i_{p,p+1}=tₚ₊₁−tₚ+dt.

After this step the detection of the heart beat and the beat to beat interval computation is finished and the algorithm waits for the next samples to arrive.

### Results

FIG. 11 is a graph showing the results obtained by the algorithm according to the invention when it is applied to a short segment of BCG data where the heart beats appear rather regularly, i.e. when no arrhythmia are present. As a reference, an ECG signal obtained at the same time as the BCG signal is plotted. By comparing the two plots, it can be seen that the algorithm clearly identifies the individual heart beats in the BCG signal.

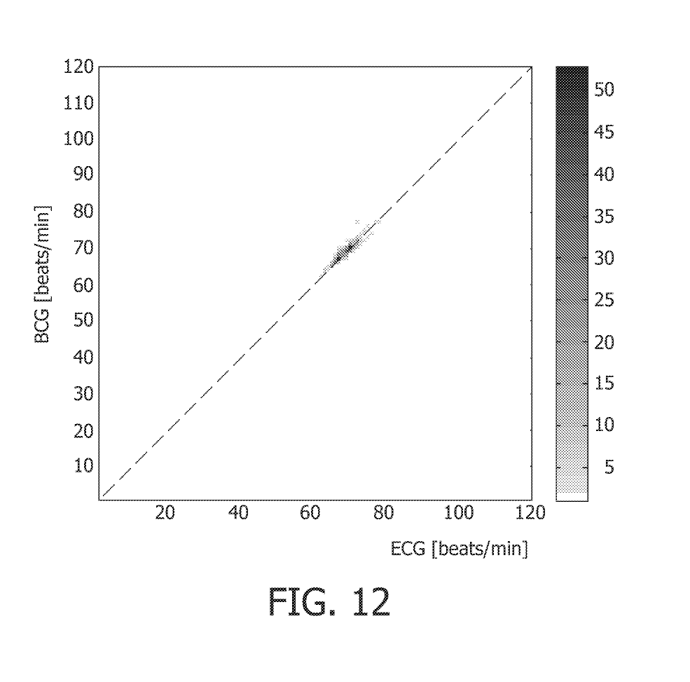

Further FIG. 12 illustrates the analysis of a BCG signal and an ECG signal that were recorded simultaneously for about 8 minutes. For each new beat the time distance to the previous beat is determined in the ECG. Likewise, the algorithm according to the invention is used to compute the corresponding beat-to-beat distances based on the BCG signal. The corresponding beat-to-beat distances of both the ECG and the BCG analysis are paired together and plotted in the scatter plot shown in FIG. 12. If both the BCG and the ECG analysis lead to identical estimates, the plot in FIG. 12 should only contain entries along the x-y diagonal (which is indicated by a dashed line). This diagonal clearly dominates FIG. 12 and only very small number of deviating pairs can be found. Averaged over the entire segment, the absolute deviation between the ECG based peak-to-peak intervals and the BCG intervals was 7 ms. Altogether only 0.6% of all peaks were missed and 0.2% false positives were detected.

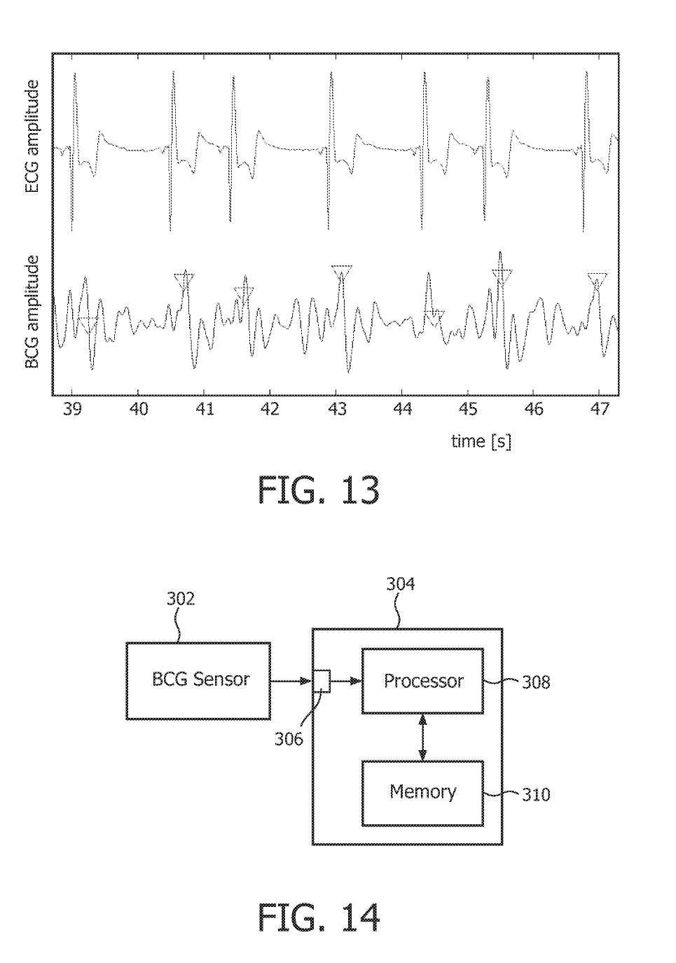

Finally, FIG. 13 shows both the ECG signal and the corresponding BCG signal of an arrythmically beating heart. As in the case of the regularly beating heart (FIG. 11) the algorithm according to the invention detects all heart beats in this segment. This is not possible with algorithms that are based on the assumption that the heart is beating regularly.

It will be appreciated by those skilled in the art that state of the art BCG sensors can be integrated invisibly into the bed of a person to be monitored and hence offer fully unobtrusive monitoring of the heart rate and also the breathing rate. Furthermore, BCG based monitoring systems are far cheaper than established intensive care unit (ICU) monitoring systems. These two features make BCG based solutions ideal for the general ward of hospitals where the patients are no longer in need of a full scale ICU monitoring system but in which the physicians are still interested if the vital functions of their patients. Especially in this setting the accurate detection of arrhythmia is important since they can often be an indicator or predictor for other severe health problems.

BCG based technologies can also be used at home for long term monitoring of the heart and breathing rate. This application is, for instance, of great interest for patients suffering from heart failure since in this case reduced heart rate variability is seen as a predictor of decompensations.

Further, especially due to its unobtrusive nature, the BCG can be used to assess sleep quality without disturbing the monitored person at night.

Although the invention has been described in terms of a method or algorithm, it will be appreciated that the invention can be implemented in a BCG system (i.e. a computer apparatus in combination with apparatus for measuring the BCG signals), or as a stand-alone computer system or program. It will be appreciated that the BCG system can provide a ballistocardiogram signal in analog or digital form to the inventive apparatus, and the inventive apparatus can be adapted to receive this signal accordingly. For example, the BCG system can provide the ballistocardiogram signal to the apparatus in analog form, and the apparatus can comprise an anti-alias filter and an analog-to-digital convertor for providing a digital representation of the ballistocardiogram signal to a suitably-programmed digital signal processor in the apparatus. Alternatively, the BCG system can implement an analog-to-digital convertor so the ballistocardiogram signal is provided to the apparatus (and specifically to a digital signal processor in the apparatus) in digital form. The apparatus can receive the ballistocardiogram signal using any appropriate means, such as through a wired or wireless connection to the BCG system.

One embodiment of an apparatus for implementing the invention is shown in FIG. 14. A ballistocardiogram signal is provided from a BCG sensor 302 to the apparatus 304. The apparatus 304 receives the BCG signal at an input port 306 and processes the BCG signal as described in the preceding description using a processor 308. Instructions for causing the processor 308 to carry out the method can be stored in a memory 310.

There is therefore provided an improved method and apparatus for detecting single heart beat events in a ballistocardiogram signal.

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality.

A single processor or other unit may fulfil the functions of several items recited in the claims. The mere fact that certain measures are recited in mutually different dependent claims does not indicate that a combination of these measured cannot be used to advantage. Any reference signs in the claims should not be construed as limiting the scope. A computer program may be stored/distributed on a suitable medium, such as an optical storage medium or a solid-state medium supplied together with or as part of other hardware, but may also be distributed in other forms, such as via the Internet or other wired or wireless telecommunication systems.

1. A method of detecting heart beats of a user in a ballistocardiogram, BCG, signal, the method comprising:
detecting heart beats in the BCG signal by locating typical features of a heart beat for the user in the BCG signal (12), the typical features of the heart beat having been obtained during a training step (10).

2. A method as claimed in claim 1, wherein the typical features of the heart beat comprise a model feature vector for a typical heart beat, c_{HBcentre}, and wherein the step of detecting heart beats comprises:
identifying characteristic points in the BCG signal (203);
determining parameters of the BCG signal at each of the identified characteristic points (204);
constituting a plurality of feature vectors from the determined parameters (205); and
using the plurality of feature vectors and c_{HBcentre} to detect heart beats in the BCG signal (206, 207, 208, 209, 210).

3. A method as claimed in claim 2, wherein the typical features of the heart beat further comprise a portion of a BCG signal used in the training step (10) corresponding to the model feature vector, s_{HBarch}, and wherein the step of using comprises:
using the plurality of feature vectors, c_{HBcentre}and s_{HBarch} to detect heart beats in the BCG signal (206, 207, 208, 209, 210).

4. A method as claimed in claim 3, wherein the step of using comprises:
computing the distance between each of the plurality of feature vectors and c_{HBcentre} (206);
identifying local minima in the resulting distances(206);
determining the cross correlation between s_{HBarch} and the BCG signal (207);
identifying local maxima in the cross correlation(207); and
identifying heart beats in the BCG signal from the locations of the local minima in the distances and the local maxima in the cross correlation (208, 209, 210).

5. A method as claimed in claim 4, wherein the step of detecting heart beats (12) further comprises:
filtering the BCG signal to obtain a high frequency component envelope (208); and
identifying maxima in the high frequency component envelope (208);
and wherein the step of identifying heart beats in the BCG signal comprises: identifying heart beats in the BCG signal from the locations of the local minima in the distances, the local maxima in the cross correlation and the local maxima in the high frequency component envelope (209, 210).

6. A method as claimed in claim 5, wherein the step of identifying heart beats in the BCG signal further comprises:
assessing the reliability of each of the local minima in the distances, the local maxima in the cross correlation and the local maxima in the high frequency component envelope (209).

7. A method as claimed in claim 6, wherein the step of identifying heart beats in the BCG signal further comprises:
forming triplets from the local minima in the distances, the local maxima in the cross correlation and the local maxima in the high frequency component envelope (210);
determining a representative value for each triplet from the assessed reliability, the representative value indicating the time at which a heart beat occurred in the BCG signal (210).

8. A method as claimed in claim 1, the method further comprising a training step (10) in which typical features of the heart beat are obtained from a portion of a BCG signal, sₜᵣₐᵢₙ, the training step comprising:
identifying characteristic points in sₜᵣₐᵢₙ (103);
determining parameters of sₜᵣₐᵢₙ at each of the identified characteristic points (104);
constituting a plurality of feature vectors from the determined parameters (105);
grouping the plurality of feature vectors into a plurality of clusters according to the similarity of the feature vectors (106);
identifying the cluster in the plurality of clusters related to the heart beats (107); and obtaining typical features of the heart beat by determining a model feature vector for a typical heart beat, c_{HBcentre} from the feature vectors in the cluster and by determining a portion of a BCG signal, s_{HBarch}, that corresponds to the model feature vector (108, 109).

9. A method as claimed in claim 8, wherein the step of identifying the cluster in the plurality of clusters related to the heart beats (107) comprises:
for each cluster in the plurality of clusters:
determining a cluster centre f_{cm} (1071);
identifying the feature vector f_{archm} that most closely matches the cluster centre f_{cm} (1072);
locating the portion s_{archm} of the BCG signal sₜᵣₐᵢₙ that corresponds to the feature vector f_{archm} (1073);
computing the distance function between the cluster centre f_{cm} and each feature vector in the cluster (1074);
computing the cross correlation of s_{archm} with the BCG signal (1075); and
determining estimates of heart beat locations in the BCG signal from feature vectors in the cluster with a local minimum for the distance function and a local maximum for the cross-correlation (1076);
filtering the BCG signal sₜᵣₐᵢₙ to identify locations of high frequency components (110); and
identifying the cluster related to the heart beat as the cluster that has:
(i) estimates of heart beat locations that are spaced by amounts that fall within a specified time window (1077); and
(ii) the local minima in the distance function and local maxima in the cross correlation that coincide best with the location of the high frequency components (1078).

10. A method as claimed in claim 1, wherein the characteristic points in the BCG signal comprise the maxima in the BCG signal.

11. A method as claimed in claim 10, wherein the parameters of each maxima point in the BCG signal comprise:
i) the amplitude (aₘₐₓ) of the maxima,
ii) the distance (dₘₐₓ) between the local maxima and the next local minima to the right,
iii) the amplitude (aₘᵢₙ) of the next local minima to the right of the local maxima, and
iv) the distance (d,ₘᵢₙ) between the next local minima to the right of the local maxima and the next local maxima to the right.

12. A method as claimed in claim 2, further comprising the step of:
reducing the dimension of the feature vectors using principal component analysis.

13. A method as claimed in claim 1, further comprising the step of:
refining the locations of heart beats detected in the BCG signal, N heart beats having been detected at times t₁, t₂, . . . , t_{N} respectively, by identifying the value of a parameter dt that maximises the cross correlation between a segment of the BCG signal between two detected heart beats at times tₚ and tₚ₊₁ respectively, where 1≦p≦N−1, and a later segment of the BCG signal between time (tₚ₊₁+dt) and time (2tₚ₊₁−tₚ+dt).

14. An apparatus (304) for use with a device (302) for measuring a ballistocardiogram signal of a user, the apparatus comprising:
means (306) for receiving a ballistocardiogram signal from the device; and
processing means (308) for performing the method defined in any one of claims 1 to 13 on the received ballistocardiogram signal.

15. A computer program product comprising computer program code that, when executed on a computer or processor, is configured to cause the computer or processor to perform the method defined in claim 1.
